(19) United States
(12) Patent Application Publication
(10) Pub. No.: US 2022/0154183 A1
(43) Pub. Date: May 19, 2022

(54) CHMP2A as a Regulator of Natural Killer Cell-Mediated Activity

(71) Applicant: The Regents of the University of California, Oakland, CA (US)

(72) Inventors: Dan S. Kaufman, La Jolla, CA (US); Davide Bernareggi, La Jolla, CA (US); Jeremy Rich, La Jolla, CA (US); Qi Xie, La Jolla, CA (US); Xiqing Li, La Jolla, CA (US); Briana Prager, La Jolla, CA (US)

(21) Appl. No.: 17/441,189

(22) PCT Filed: Mar. 23, 2020
(86) PCT No.: PCT/US2020/024218

§ 371 Date: Sep. 20, 2021

Related U.S. Application Data
(60) Provisional application No. 62/822,259, filed on Mar. 22, 2019.

(51) Int. Cl. C12N 15/113; A61K 31/4709

(52) U.S. Cl. CPC C12N 15/113; A61K 31/4709; C12N 2320/30; C12N 2310/20; C12N 2310/14

(57) ABSTRACT

Methods and compositions for increasing tumor cell sensitivity to natural killer cell-mediated toxicity by inhibiting CHMP2A or other endosomal sorting complex required for transport in the tumor cell.

### CROSS REFERENCE TO RELATED APPLICATIONS

This application claims the priority benefit of U.S. Provisional Application No. 62/822,259, filed Mar. 22, 2019, which application is incorporated herein by reference.

### GOVERNMENT SPONSORSHIP

This invention was made with government support under grant No. CA217885 awarded by the National Institutes of Health. The government has certain rights in the invention.

### TECHNICAL FIELD

The present invention relates to treatments for glioblastomas, head and neck squamous cell cancer, and other tumors.

### BACKGROUND

CHMP2A is known to be part of the endosomal sorting complex required for transport (ESCRT)⁽¹⁾ and impairment of CHMP2A function can block the entire complex. ESCRTIII is involved in multi-vesicular bodies (MVB) formation and exosome formation and secretion. Exosomes derived from tumor cells can act as immune cell inhibitors and can have immune suppressive effects on NK cells and T cells.^{(2,20,21)} ESCRTIII can be involved in cell membrane repair⁽⁷⁾ and in blocking NFkB,⁽⁸⁾ a transcription factor that drives the inflammation process through the synthesis of cytokines and chemokines.

The current standard of care for patients with glioblastoma includes surgery, temozolomide chemotherapy, radiotherapy, and corticosteroids, all of which have immunosuppressive effects.^{(3,4)} Increasing the sensitivity of GSC to NK cell immunotherapy may improve treatment following surgery or radiotherapy that can leave in a patient's brain a few dispersed cells that can form new tumors after removal.

Glioblastoma (GBM) is the most common and most aggressive primary malignant brain tumor in adults. The overall annual incidence of gliomas in the USA is ˜6 cases per 100,000 individuals, with glioblastoma accounting for ˜50% of cases, and the disease has a male predominance.⁽⁵⁾ GBM is a highly suppressive tumor and the interaction with NK cells results in suppression of their activity, which is mediated by atypical HLA molecules (including HLA-E and HLA-G).⁽⁵⁾ The standard treatments for newly diagnosed GBM, the combination of radiation therapy (RT) and alkylating chemotherapy, confounds this immunosuppression. Often steroids are necessary for management of peritumoral edema, but they decrease the efficacy of immunotherapies. No FDA-approved immunotherapy for GBM exists, despite its long history of Immunotherapy, including immune stimulation, antibody-mediated immunotherapies, adoptive cellular immunotherapies, and vaccines. Ongoing clinical research for the treatment of GBM includes checkpoint inhibitor antibodies, vaccines, CAR-T cell therapy, and oncolytic viral therapy.⁽³⁾

Head and neck squamous cell carcinoma (HNSCC) is the sixth most common form of cancer worldwide and it accounts for more than 90% of the cancers of the head and neck. 600,000 people worldwide are diagnosed for HNSCC, with 50,000 new cases and 10,000 deaths occurring in the United States.⁽¹⁷⁾ Treatments typically consist of surgery, radiotherapy and chemotherapy—all associated with significant morbidity and mortality. The overall long-term survival remains low due to persistent or recurrent disease with only 40-50% of patients surviving more than five years.⁽¹⁷⁾ New antibody-based treatments have added to the armamentarium against HNSCC.⁽¹⁸⁾ Cetuximab, a monoclonal antibody that inhibits EGFR, is a cancer targeting drug approved for HNSCC. Unfortunately, only 10-15% of patients respond to cetuximab as monotherapy with progression-free survival of 5.6 months.⁽¹⁷⁾ Nivolumab and pembrolizumab, two new immune check point inhibitors targeting PD-1, have recently been approved for HNSCC treatment, showing an immune modulation and durable remission even if presenting a response in only 20% of patients,⁽¹⁹⁾ lower than other tumor types like melanoma.

### SUMMARY OF THE INVENTION

The invention describes a novel pathway that increases the sensitivity of tumor cells, such as glioblastoma, to natural killer (NK) cell-mediated cytotoxicity by blocking the transcription of the genes, such as CHMP2A, to impair the synthesis of the functional protein.

The invention provides pharmaceutical compositions and methods of treatment that can block ESCRT by acting on CHMP2A or other protein parts of the complex through acting on the DNA, RNA or protein level.

CHMP2A and the ESCRT complex are not previously known to be potential targets to regulate NK cells or other immune cell immunotherapy. This invention shows that blocking their function increases GSC and HNSCC sensitivity to NK cells. The invention also provided methods of screening molecules and compositions for identification of those that can improve immunotherapy by acting on ESCRT complexes and/or on their components (like CHMP2A).

The present invention provides methods and compositions for treating a tumor in a subject comprising administering to a subject having a tumor in need thereof an effective amount of a pharmaceutically acceptable composition comprising an agent that inhibits CHMP2A gene expression or function, or the endosomal sorting complex required for transport (ESCRT), in a cell of the tumor.

In embodiments, the agent inhibits transcription or translation of CHMP2A gene. In embodiments, the agent is a shRNA that inhibits CHMP2A gene expression. In embodiments, the agent inhibits ESCRT. In embodiments, the agent is a farnesyltransferase inhibitor, including tipifarnib. In embodiments, the agent inhibits transcription or translation of CHMP2A gene. In embodiments, the agent inhibits transcription or translation of PTPN9 gene.

In embodiments, the administration increases tumor sensitivity to natural killer cells. In embodiments, the tumor is a glioblastoma, meningioma or head and neck squamous cell carcinoma (HNSCC).

The present invention provides methods and compositions for treating a tumor in a subject comprising administering to a subject having a tumor in need thereof an effective amount of a pharmaceutically acceptable composition comprising an agent that inhibits gene expression or function of CHMP2A, PTPN9, ACER1, SNX7, MSR1, ANKRD46, IFT81, PLEKHF2, TRMT10A, or NARS in a cell of the tumor.

In embodiments, the agent inhibits transcription or translation of CHMP2A gene. In embodiments, the agent is a shRNA that inhibits CHMP2A gene expression. In embodiments, the agent inhibits ESCRT. In embodiments, the agent is a farnesyltransferase inhibitor, including tipifarnib. In embodiments, the agent inhibits transcription or translation of CHMP2A gene. In embodiments, the agent inhibits transcription or translation of PTPN9 gene.

In embodiments, the administration increases tumor sensitivity to natural killer cells. In embodiments, the tumor is a glioblastoma, meningioma or head and neck squamous cell carcinoma (HNSCC).

The present invention provides pharmaceutical compositions comprising an agent that inhibits CHMP2A gene expression or function, or the endosomal sorting complex required for transport (ESCRT), in a cell of the tumor for use in treating the tumor in a patient in need thereof.

The present invention provides a pharmaceutical composition comprising an agent that inhibits gene expression or function of CHMP2A, PTPN9, ACER1, SNX7, MSR1, ANKRD46, IFT81, PLEKHF2, TRMT10A, and NARS, in a cell of the tumor for use in treating the tumor in a patient in need thereof. In embodiments, the patient has a tumor cell is a glioblastoma, meningioma or head and neck squamous cell carcinoma (HNSCC).

The present invention provides methods of increasing cell-mediated killing immune response capacity of natural killer (NK) cells in a subject in need thereof, comprising administering to the subject an effective amount of an agent that inhibits gene expression or function of CHMP2A, PTPN9, ACER1, SNX7, MSR1, ANKRD46, IFT81, PLEKHF2, TRMT10A, and NARS. In embodiments, the tumor cell is a glioblastoma, meningioma or head and neck squamous cell carcinoma (HNSCC).

The present invention provides methods of screening a molecule or composition for therapeutic candidates, comprising administering the molecule or composition to a tumor model to identify those candidates that increase tumor sensitivity to natural killer cells. In embodiments, the method identifies molecules or compositions that inhibit gene expression or function of CHMP2A, PTPN9, ACER1, SNX7, MSR1, ANKRD46, IFT81, PLEKHF2, TRMT10A, or NARS, or the ESCRT, in the tumor model.

### BRIEF DESCRIPTION OF THE DRAWINGS

FIG. 1 shows a possible mechanism of action of blocking CHMP2A and the formation of ESCRT.

FIG. 2 shows the experimental procedure of the TCT screening on GSC cells and NK cells.

FIG. 3 shows Casp3/7 NK killing assay performed on GSC lines (CW468, D456, 387).

FIG. 4 shows Casp3/7 NK killing assay performed on GSC 387 using conditioned media from CHMP2A KO (sgRNA #2) or the Mock cell line.

FIGS. 5A-5C show a Casp3/7 NK killing assay performed on HNSCC cell lines.

FIG. 6 shows genes upregulated in GSC lines KO for CHMP2A.

FIGS. 7A-7C show that NK cells increase migration towards cells KO for CHMP2A.

FIG. 8 shows Cal27 CHMP2A KO cells produce less EV than a mock cell line and have bigger EV on average.

FIG. 9 shows Cal27 CHMP2A KO cells have an increased sensitivity to NK cell-mediated killing in a HNSCC subcutaneous xenograft mouse model.

FIG. 10 shows that Tipifarnib treated Cal27 cells show an increased sensitivity to NK cells mediated killing.

FIG. 11 shows CW465 cells increased sensitivity to PB-NK cell killing when PTPN9 is not expressed

### DETAILED DESCRIPTION

Exosomes derived from tumor cells can act as immune cell inhibitors and have immune suppressive effects on NK cells. CHMP2A is known to be part of the endosomal sorting complex required for transport (ESCRT) and impairment of this function can block the entire complex. CHMP2A is pan of the ESCRT machinery that enables formation of multi-vesicular bodies, which contain exosomes. Blocking exosome function can increase GSC gene sensitivity to NK cells and other immune cells, with the use of drugs that can improve immunotherapy by acting on ESCRT complexes or ESCRT components (like CHMP2A) or other proteins part of the complex acting on DNA, RNA or protein level to increase the sensitivity of glioblastoma and other tumors to NK cell-mediated killing. These drugs can be used either alone or in combination with other modalities or adoptive transfer of NK cell-based immunotherapy for improved anti-tumor activity.

Specifically, ESCRTIII is a complex of proteins involved in multi-vesicular bodies (MVB) and exosome formation and secretion. This invention shows that blocking of one of its components, CHMP2A, leads tumors to have higher susceptibility to NK cells. CHMP2A can be blocked by inhibiting its gene or by blocking CHMP2A integration in the ESCRTIII complex by inhibiting its RNA or by blocking the protein itself. The ESCRTIII complex can be inhibited by acting on other components of the complex. For example, ESCRTIII can be impaired by knock-out (KO) of CHMP4⁽⁸⁾ as well as by KO of other proteins in the complex. This will block the release of extracellular vesicles (EV) like exosomes or microvesicles that can inhibit NK cell killing by transferring some inhibitory small RNA molecules or by binding and saturating the activating ligand on NK cells that can became exhausted.⁽⁹⁾ CHMP2A KO and subsequent blocking of ESCRTIII can increase the inflammation and chemokines and cytokines released through NFkB activation.⁽⁸⁾ More chemokines recruit more NK cells increasing the overall killing potential. Since ESCRTIII is involved in cell membrane repair, by KO CHMP2A with the following inactivation of ESCRTIII, cells damaged from the release of granules by NK cells are more susceptible to cell death lacking one of the cell membrane repair mechanisms.⁽⁷⁾ This will lead to an increased sensitivity of GSC to NK cells killing, reducing the number of proliferative cells in glioblastoma. Because the ESCRT complexes as a whole are involved in the mechanisms of extracellular vesicles release, impairing other ESCRT complexes besides ESCRTIII may lead to similar results.

Moreover, because ESCRTIII has been implicated in the packaging of HIV⁽¹⁰⁾ and is generally involved in virus budding, blocking the protein complex as described in this invention provides therapies outside the field of oncology. For example, the invention can block the release of some RNA viruses like Ebola, or coronaviruses.⁽¹¹⁾ On the other hand, since ESCRTII is also important in cytokinesis (one step of cell division) an up-regulation can increase or make more efficient cell replication which is applicable in cell regeneration and tissue repair.

The present invention provides methods and compositions for treating a tumor in a subject comprising administering to a subject having a tumor in need thereof an effective amount of a pharmaceutically acceptable composition comprising an agent that inhibits CHMP2A gene expression or function, or the endosomal sorting complex required for transport (ESCRT), in a cell of the tumor.

In embodiments, the agent inhibits transcription or translation of CHMP2A gene. In embodiments, the agent is a shRNA that inhibits CHMP2A gene expression. In embodiments, the agent inhibits ESCRT. In embodiments, the agent is a farnesyltransferase inhibitor, including tipifarnib. In embodiments, the agent inhibits transcription or translation of CHMP2A gene. In embodiments, the agent inhibits transcription or translation of PTPN9 gene.

In embodiments, the administration increases tumor sensitivity to natural killer cells. In embodiments, the tumor is a glioblastoma, meningioma or head and neck squamous cell carcinoma (HNSCC).

The present invention provides methods and compositions for treating a tumor in a subject comprising administering to a subject having a tumor in need thereof an effective amount of a pharmaceutically acceptable composition comprising an agent that inhibits gene expression or function of CHMP2A, PTPN9, ACER1, SNX7, MSR1, ANKRD46, IFT81, PLEKHF2, TRMT10A, or NARS in a cell of the tumor.

In embodiments, the agent inhibits transcription or translation of CHMP2A gene. In embodiments, the agent is a shRNA that inhibits CHMP2A gene expression. In embodiments, the agent inhibits ESCRT. In embodiments, the agent is a farnesyltransferase inhibitor, including tipifarnib. In embodiments, the agent inhibits transcription or translation of CHMP2A gene. In embodiments, the agent inhibits transcription or translation of PTPN9 gene.

In embodiments, the administration increases tumor sensitivity to natural killer cells. In embodiments, the administration increases tumor sensitivity to other immune cells, such as T cells and cytotoxic T cells, and increases other immune cell-mediated activity. In embodiments, the tumor is selected from but not limited to a glioblastoma, meningioma or head and neck squamous cell carcinoma (HNSCC).

The present invention provides pharmaceutical compositions comprising an agent that inhibits CHMP2A gene expression or function, or the endosomal sorting complex required for transport (ESCRT), in a cell of the tumor for use in treating the tumor in a subject in need thereof.

The present invention provides a pharmaceutical composition comprising an agent that inhibits gene expression or function of CHMP2A, PTPN9, ACER1, SNX7, MSR1, ANKRD46, IFT81, PLEKHF2, TRMT10A, and NARS, in a cell of the tumor for use in treating the tumor in a subject in need thereof. In embodiments, the tumor is a glioblastoma, meningioma or head and neck squamous cell carcinoma (HNSCC).

The present invention provides methods of increasing cell-mediated killing immune response capacity of immune cells, such as natural killer (NK) cells, in a subject in need thereof, comprising administering to the subject an effective amount of an agent that inhibits gene expression or function of CHMP2A, PTPN9, ACER1, SNX7, MSR1, ANKRD46, IFT81, PLEKHF2, TRMT10A, and NARS. In embodiments, the tumor cell is a glioblastoma, meningioma or head and neck squamous cell carcinoma (HNSCC).

In embodiments, the agent of the pharmaceutical compositions is a small molecule (e.g. a farnesyltransferase inhibitor), nucleic acid (e.g., a shRNA or other RNAi) or protein (e.g., an antibody). In embodiments, the agent inhibits transcription or translation of CHMP2A, PTPN9, ACER1, SNX7, MSR1, ANKRD46, IFT81, PLEKHF2, TRMT10A, or NARS gene. In embodiments, the agent is a shRNA that inhibits CHMP2A, PTPN9, ACER1, SNX7, MSR1, ANKRD46, IFT81, PLEKHF2, TRMT10A, or NARS gene expression. In embodiments, the agent inhibits ESCRT. In embodiments, the agent is a farnesyltransferase inhibitor, including tipifarnib. In embodiments, the agent inhibits transcription or translation of CHMP2A gene. In embodiments, the agent inhibits transcription or translation PTPN9 gene. In embodiments, the agent increases tumor sensitivity to natural killer cells. In embodiments, the tumor is a glioblastoma, meningioma or head and neck squamous cell carcinoma (HNSCC).

The present invention provides methods of screening a molecule or composition for to identify candidates for tumor therapy, comprising administering the molecule or composition to a tumor model to identify those candidates that increase tumor sensitivity to natural killer cells. In embodiments, the method identifies molecules or compositions that inhibit gene expression or function of CHMP2A, PTPN9, ACER1, SNX7, MSR1, ANKRD46, IFT81, PLEKHF2, TRMT10A, or NARS, or the ESCRT, in the tumor model. In embodiments, the invention provides a two cell type screening as described herein to identify novel key molecular targets and mechanisms that lead to a better targeting and killing of glioblastoma stem cells (GSC) and meningioma cells by NK cells.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See. e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” or “effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to age-related eye diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable or constant region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies. i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

The invention may also refer to any oligonucleotides (antisense oligonucleotide agents), polynucleotides (e.g. therapeutic DNA), ribozymes, DNA aptamers, dsRNAs, siRNA, shRNA, RNAi, and/or gene therapy vectors. The term “antisense oligonucleotide agent” refers to short synthetic segments of DNA or RNA, usually referred to as oligonucleotides, which are designed to be complementary to a sequence of a specific mRNA to inhibit the translation of the targeted mRNA by binding to a unique sequence segment on the mRNA. Antisense oligonucleotides are often developed and used in the antisense technology. The term “antisense technology” refers to a drug-discovery and development technique that involves design and use of synthetic oligonucleotides complementary to a target mRNA to inhibit production of specific disease-causing proteins. Antisense technology permits design of drugs, called antisense oligonucleotides, which intervene at the genetic level and inhibit the production of disease-associated proteins. Antisense oligonucleotide agents are developed based on genetic information.

As an alternative to antisense oligonucleotide agents, ribozymes or double stranded RNA (dsRNA), short hairpin RNA (shRNA), RNA interference (RNAi), and/or small interfering RNA (siRNA), can also be used as therapeutic agents for regulation of gene expression in cells. As used herein, the term “ribozyme” refers to a catalytic RNA-based enzyme with ribonuclease activity that is capable of cleaving a single-stranded nucleic acid, such as an mRNA, to which it has a complementary region. Ribozymes can be used to catalytically cleave target mRNA transcripts to thereby inhibit translation of target mRNA. The term “dsRNA,” as used herein, refers to RNA hybrids comprising two strands of RNA. The dsRNAs can be linear or circular in structure. The RNA may comprise ribonucleotides, ribonucleotide analogs, such as 2′-O-methyl ribosyl residues, or combinations thereof. The term “RNAi” refers to RNA interference or post-transcriptional gene silencing (PTGS). The term “siRNA” refers to small dsRNA molecules (e.g., 21-23 nucleotides) that are the mediators of the RNAi effects. RNAi is induced by the introduction of long dsRNA (up to 1-2 kb) produced by in vitro transcription, and has been successfully used to reduce gene expression in variety of organisms. In mammalian cells, RNAi uses siRNA (e.g. 22 nucleotides long) to bind to the RNA-induced silencing complex (RISC), which then binds to any matching mRNA sequence to degrade target mRNA, thus, silences the gene. A short hairpin RNA or small hairpin RNA (shRNA/Hairpin Vector) is an artificial RNA molecule with a tight hairpin turn that can be used to silence target gene expression via RNA interference (RNAi). Expression of shRNA in cells is typically accomplished by delivery of plasmids or through viral or bacterial vectors. Single guide RNA (sgRNA) as used in a CRISPR gene editing system is a single RNA molecule that contains both the custom-designed short crRNA sequence fused to the scaffold tracrRNA sequence. The RNAs used in the invention are based on publicly known sequences for the targeted gene and can be synthetically generated or made in vitro or in vivo from a DNA template, and are commercially available.

“Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

A nucleic acid is typically single-stranded or double-stranded and will generally contain phosphodiester bonds, although in some cases, as outlined, herein, nucleic acid analogs are included that may have alternate backbones, including, for example and without limitation, phosphoramide (Beaucage et al. (1993) Tetrahedron 49(10):1925 and references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26: 1419, which are each incorporated by reference), phosphorothioate (Mag et al. (1991) Nucleic Acids Res. 19:1437; and U.S. Pat. No. 5,644,048, which are both incorporated by reference), phosphorodithioate (Briu et al. (1989) J. Am. Chem. Soc. 111:2321, which is incorporated by reference), O-methylphosphoroamidite linkages (see Eckstein, Oligonucleotides and Analogues: A Practical Approach, Oxford University Press (1992), which is incorporated by reference), and peptide nucleic acid backbones and linkages (see, Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207, which are each incorporated by reference). Other analog nucleic acids include those with positively charged backbones (Denpcy et al. (1995) Proc. Natl. Acad. Sci. USA 92:6097, which is incorporated by reference); non-ionic backbones (U.S. Pat. Nos. 5,386.023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem: Lett. 4: 395; Jeffs et al. (1994) J. Biomolecular NMR 34:17; and Tetrahedron Lett. 37:743 (1996), which are each incorporated by reference) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (see Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labels, or to alter the stability and half-life of such molecules in physiological environments.

### Examples

Natural killer (NK) cells play a key role in tumor immune-surveillance by their ability to recognize and kill both hematological malignancies and solid tumor cells. NK cell-based immunotherapy is emerging as an important cancer treatment; however, selective pressures can lead tumor cells to develop resistance mechanisms to escape NK cell-mediated killing by modulating expression of specific genes involved in the interaction between tumor and immune cells. This example uses a novel “two cell type” screening to identify novel key molecular targets and mechanisms that lead to a better targeting and killing of glioblastoma stem cells (GSC) and meningioma cells by NK cells. To mimic loss of function mutations involved in resistance or sensitivity to NK cells, the example uses a genome-scale CRISPR-Cas9 library to induce mutations in 19,000 genes in four human GSC lines and two meningioma cell lines. After a round of selection with peripheral blood NK cells, the genes that impaired the killing activity of NK cells or increased the sensitivity of tumor cells to NK cells were profiled. The most common genes lost in GSC and meningioma cells that increase their sensitivity to NK cells are involved in the ER-phagosome pathway, antigen presentation and cellular localization. One gene of interest is a component of a complex involved in degradation of surface receptor proteins and formation of endocytic multivesicular bodies. This invention confirms that impairing its function in GSC lines can increase their sensitivity to NK cell-mediated killing. RNA sequencing demonstrates that it increases the expression of genes involved in response to the adaptive immune system, and that chemokines and cytokines overexpressed by the KO cells more effectively stimulate NK cell-mediated killing of these tumor cells.

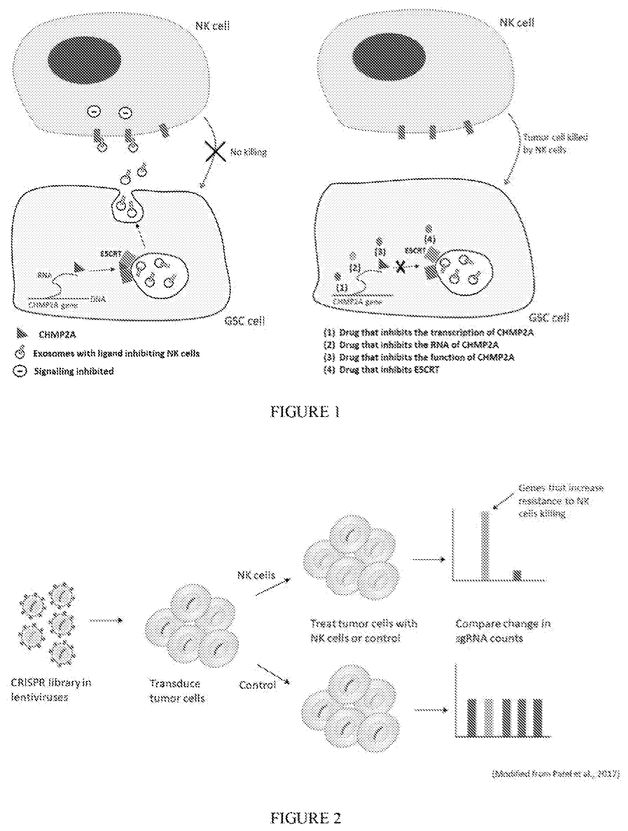

FIG. 1 shows the possible mechanism of action of blocking CHMP2A and the formation of ESCRT. ESCRT is a complex of proteins involved in MVB and exosome formation and secretion. This invention acts by blocking one of its components, CHMP2A by blocking its gene. CHMP2A integration in the ESCRT complex may also be blocked by inhibiting its RNA or by blocking the protein itself. The ESCRT complex may also be inhibited by acting on other components associated therewith. This will block the release of vesicles like exosomes that can carry on their surface inhibiting ligands for NK cells (FIG. 1). This will lead to an increased sensitivity of GSC to NK cell killing, thereby reducing the number of proliferative cells in glioblastoma tumors.

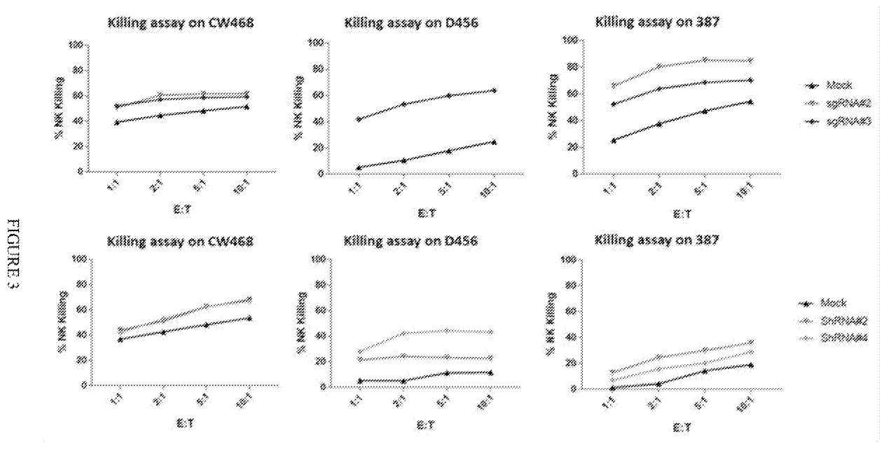

FIG. 2 shows the experimental procedure of the TCT screening on GSC cells and NK cells. Genomic data from transduced GSC co-incubated with NK cells are compared with transduced cells without NK cells to analyze what genes may be involved in NK cell-mediated killing sensitivity or resistance. First, a whole genome CRISPR/CAS9 KO was performed to screen four lines of GSC. After selection, the GSCs were incubated with NK cells for 24 hours harvesting tumor cells 24 hours later. DNA sequencing on the GSC lines was performed to discover what gRNAs were most enriched in the population of cells more resistant to NK cell killing. Less frequent gRNAs were associated with those genes that increase sensitivity to NK cell killing. Genes lost in NK-sensitive cells are enriched in ER-phagosome pathway, antigen presentation and cellular localization.

A plot of the genetic analysis of TCT screen for genetic mediators of NK cell-mediated killing of GSC was created (not shown). The top 20 hit genes expressed in the 4 GSC lines that give resistance or sensitivity to NK cell-mediated killing were averaged. The first 10 genes that increased sensitivity to NK cells were, in order, CHMP2A, PTPN9, ACER1, SNX7, MSR1, ANKRD46, IFT81, PLEKHF2, TRMT10A, and NARS.

Because CHMP2A had the highest score, it was validated on GSCs as a single KO in three cell lines and cells showed increased sensitivity to NK cell mediated killing (FIG. 3). CHMP2A was silenced using shRNAs to confirm its role in GSC lines (FIG. 3). Specifically, FIG. 3 shows Casp3/7 NK killing assay performed on GSC lines (CW468, D456, 387). Two sgRNA (#2, #3, top row) and two shRNA (#2, #4, bottom row), were used to validate CHMP2A KO. GSC showed increased sensitivity to PB-NK cell killing when CHMP2A is KO. E:T=Effector to Target ratio.

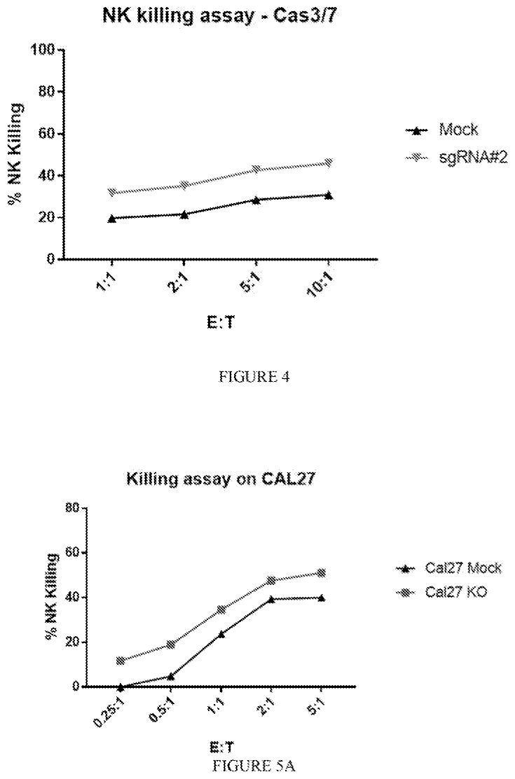

The function of immune cells can be impaired by exosomes released by tumor cells⁽²⁾ and CHMP2A is part of the ESCRT machinery that enable the formation of MVB⁽¹⁾ that can contain exosomes. Therefore, the conditioned media from the cell line GSC 387 KO for CHMP2A was used to incubate PB-NK cells for 1 hour and perform a killing assay on the GSC 387 wild type cell line. The media conditioned by GSC 387 Mock line was used as a control. These results demonstrate an increased sensitivity to NK cell-mediated killing in tumor cells where the media from CHMP2A KO cells was used (FIG. 4). Specifically, FIG. 4 shows a Casp3/7 NK killing assay performed on GSC 387 using conditioned media from CHMP2A KO (sgRNA #2) or the Mock cell line. Cells incubated with KO conditioned media showed an increased sensitivity to PB-NK cells. This experiment provides evidence that GSC KO for CHMP2A are unable to secrete some factors or exosomes that can reduce NK cells killing.

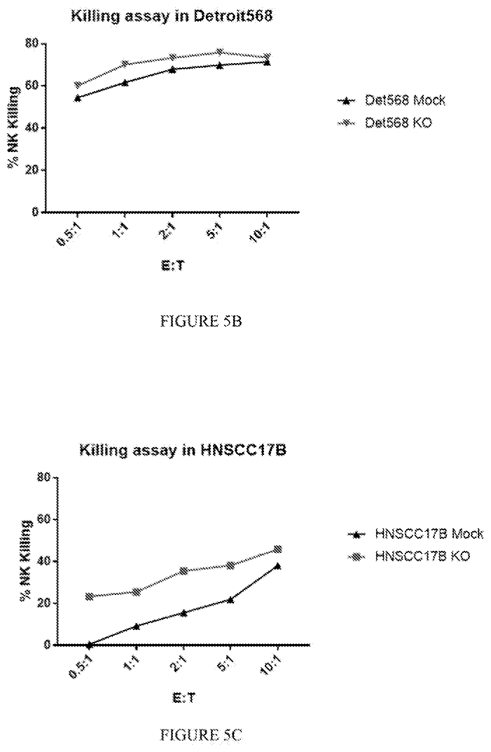

The KO of CHMP2A was also tested in some head and neck squamous cells carcinoma (HNSCC) cell lines showing an increased sensitivity to NK-mediated killing like for GSC (FIGS. 5A-5C). Specifically, in FIGS. 5A-5C a Casp3/7 NK killing assay was performed on HNSCC cell lines (Cal27. Detroit568 and HNSCC17B). sgRNA targeting CHMP2A was used to validate CHMP2A KO. HNSCC cells showed increased sensitivity to PB-NK cell killing when CHMP2A is KO. E:T=Effector to Target ratio.

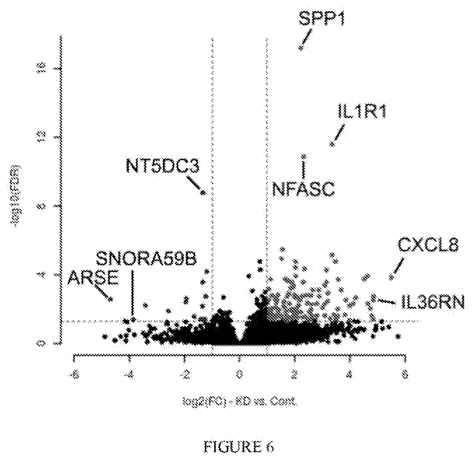

Performing the RNA sequencing in CHMP2A KO cells showed a pattern of upregulated genes involved in tumor cell-immune cell interactions and inflammation with some chemokines and cytokines upregulated (FIG. 4). FIG. 6 shows genes upregulated in GSC lines KO for CHMP2A. Some of the most upregulated genes were chemokines or genes related to interferon gamma response and inflammation, as shown in Table 1.

TABLE 1
Genes upregulated in CHMP2A KO GSC cells (GSC 387, CW465)

| | |
|---|---|
| CXCL8 (IL8) | IL32 |
| C3 | CCL3 |
| CXCL2 | IFIT1 (interferon-induced protein with |
| | tetratricopeptide repeats 1) |
| CCL20 | IFIT2 (interferon-induced protein with |
| | tetratricopeptide repeats 1) |
| CXCL10 | CXCL12 |
| IL36G | RUNX3 (runt-related transcription factor 3) |

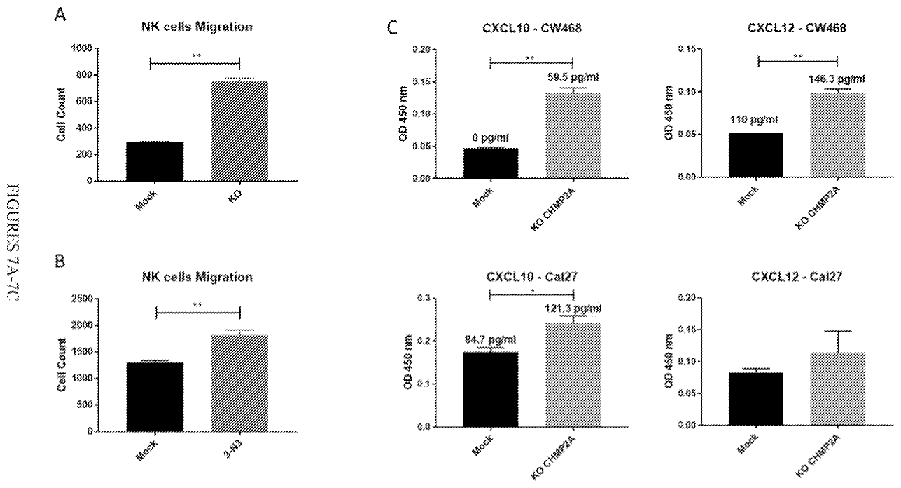

ESCRTIII and also the other ESCRT complexes function to suppress spontaneous NFkB activation in 293 cells.⁽⁸⁾ NFkB can induce inflammation and therefore increase the secretion of inflammatory cytokines and chemokines like CXCL10 and CXCL12.^{(12,13)} Overexpression of CXCL10 or its endogenous application in a tumor increases NK cells migration towards the tumor site⁽¹⁴⁾ and, in general, CXCL10 and CXCL12 are important regulators of NK cells migration.⁽¹⁵⁾ Therefore, the dysregulation of ESCRTIII through the KO of CHMP2A can increase NK cells migration towards tumor cells, increasing the number of NK cells that can kill tumor cells. This increased NK cell migration towards cancer cells was observed through a migration assay using CHMP2A KO cells as chemoattractant (FIGS. 7A-7B). These chemokines were also found in the conditioned media from KO cells (FIG. 7C). Specifically, FIGS. 7A-7B show that NK cells increase their migration towards cells KO for CHMP2A in both GSC CW465 (FIG. 7A) and HNSCC Cal27 (FIG. 7B). CHMP2A KO cells produce more CXCL10 and CXCL12 in their media meaning that this can be a possible mechanism that increases NK cells migration and subsequent killing (FIG. 7C).

CHMP2A KO Cal27 showed a lower number of EV and a different size from the mock cells, meaning interference in the release of EV was realized (FIG. 8). This may be another mechanism of action of CHMP2A as described in FIG. 1.

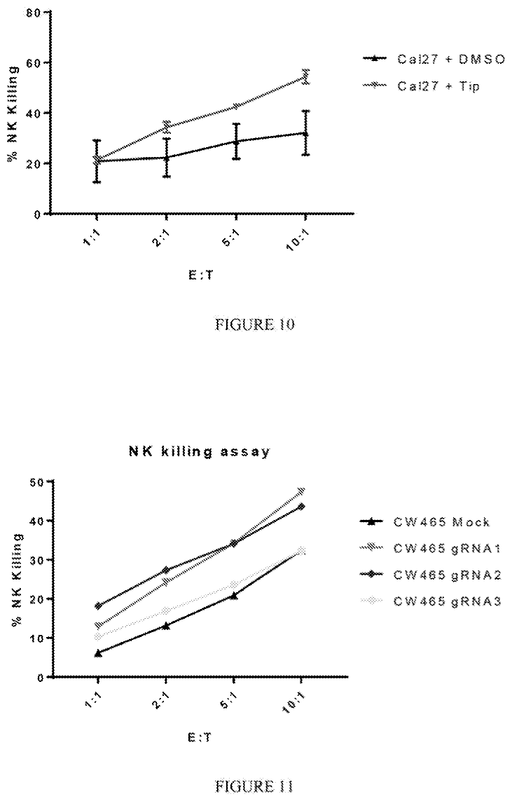

In order to demonstrate the importance of the ESCRTIII complex and its function in determining the sensitivity of tumor cells to NK cells-mediated killing, wild type Cal27 cells were treated with the farnesyl transferase inhibitor, Tipifarnib, at a concentration of 1 μM for 72 hours. Tipifarnib has been shown to inhibit the release of EV and exosomes specifically by acting on the ESCRTIII-dependent pathway.⁽¹⁶⁾ Tipifarnib treated Cal27 cells showed an increased sensitivity to NK cells mediated killing (FIG. 10) demonstrating the importance of ESCRTIII and EV release in NK cell-mediated killing increased sensitivity.

Specifically, in FIG. 10, Cal27 wild type cells were treated with 1 μM of Tipifarnib (Tip) for 72 hours and a control was treated with DMSO. Tipifarnib treated cells showed an increased sensitivity to NK cells killing in a Casp3/7 plus SytoxAAD killing assay.

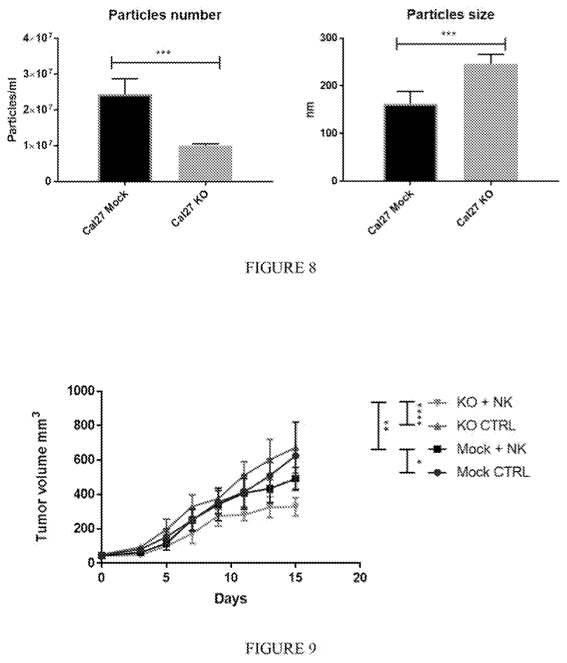

Using an in vivo xenograft mouse model for HNSCC (Cal27 cells) it was demonstrated that blocking CHMP2A increased the efficacy of NK cell treatment in a mouse model of a solid tumor (FIG. 9). Specifically, in FIG. 9, Cal27 CHMP2A KO showed an increased sensitivity to NK cell-mediated killing in a HNSCC subcutaneous xenograft mouse model. By releasing more chemokines (CXCL10 and CXCL12), Cal27 KO increased NK cell migration towards the tumor site and by producing less exosomes reduced the inhibiting signal to NK cells killing.

CHMP2A and the ESCRTIII pathway inhibition is an important target for solid tumors and can be used to increase NK cell-based immunotherapy. Moreover, since CHMP2A was one of the top hits in the same screening on meningioma cell lines, meningioma cells could also be sensitive. TCGA data show that in some tumor types low CHMP2A expression confers a better survival, even if not statistically significant.

This invention can be used to screen for and develop new drugs able to increase the sensitivity of tumor cells to NK cell-mediated killing. Such drugs can be used in combination with NK cell immunotherapy (such as ESC or iPSC derived NK cells) to improve outcomes.

It is noted that although CHMP2A had the highest score, it is not the only gene that can affect NK cell sensitivity. For example, the validation in vitro of PTPN9, another promising target from the screening on GSC, showed the importance of a “two cell type” screening involving tumor cells and NK cells for finding new promising targets to increase tumor sensitivity to NK cells-mediated killing. This screening can be also applied using pluripotent stem cells (iPSC) derived NK cells as effectors.

FIG. 11 shows a Casp3/7 plus SytoxAAD NK killing assay performed on CW465 GSC line. Four sgRNA targeting PTPN9 were used to validate PTPN9 KO. CW465 cells showed increased sensitivity to PB-NK cell killing when PTPN9 is not expressed.

### REFERENCES

1. Tsang H T, Connell J W, Brown S E, Thompson A, Reid E, Sanderson C M. A systematic analysis of human CHMP protein interactions: additional MIT domain-containing proteins bind to multiple components of the human ESCRT III complex. Genomics. 2006; 88(3):333-46.
2. Reiners K S, Dassler J, Coch C, Pogge von Strandmann E. Role of Exosomes Released by Dendritic Cells and/or by Tumor Targets: Regulation of NK Cell Plasticity. Frontiers in immunology. 2014:5:91.
3. McGranahan T, Therkelsen K E, Ahmad S, Nagpal S. Current State of Immunotherapy for Treatment of Glioblastoma. Current treatment options in oncology. 2019; 20(3):24
4. Fernandes C C A, Osorio L, et al., Current Standards of Care in Glioblastoma Therapy. Glioblastoma. 2017; Chapter 11.
5. Lim M, Xia Y, Bettegowda C, Weller M. Current state of immunotherapy for glioblastoma. Nature reviews Clinical oncology. 2018; 15(7):422-42.
6. Kalluri R, LeBleu V S. The biology, function, and biomedical applications of exosomes. Science. 2020 Feb. 7; 367(6478). pii: eaau6977. doi: 10.1126/science.aau6977.
7. Gong Y N, Guy C, Olauson H, Becker J U, Yang M, Fitzgerald P, Linkermann A, Green D R. ESCRT-III Acts Downstream of MLKL to Regulate Necroptotic Cell Death and Its Consequences. Cell. 2017 Apr. 6; 169(2):286 300.e16. doi: 10.1016/j.cell.2017.03.020.
8. Mamińska A, et al., ESCRT proteins restrict constitutive NF-KB signaling by trafficking cytokine receptors. Sci Signal. 2016 Jan. 19; 9(411):ra8. doi: 10.1126/scisignal.aadU848.
9. Maia J, Caja S, Strano Moraes M C, Couto N, Costa-Silva B. Exosome-Based Cell-Cell Communication in the Tumor Microenvironment. Front Cell Dev Biol. 2018 Feb. 20; 6:18. doi: 10.3389/fcell.2018.00018. eCollection 2018.
10. Morita E, Sandrin V, McCullough J, Katsuyama A, Baci Hamilton I, Sundquist W I. ESCRT-III protein requirements for HIV-1 budding. Cell Host Microbe. 2011 Mar. 17; 9(3):235-242. doi: 10.1016/j.chom.2011.02.004.
11. Votteler J, Sundquist W I. Virus budding and the ESCRT pathway. Cell Host Microbe. 2013 Sep. 11; 14(3):232-41. doi: 10.1016/j.chom.2013.08.012.
12. Jin W J, Kim B, Kim D, Park Choo H Y, Kim H H, Ha H, Lee Z H. NF-κB signaling regulates cell-autonomous regulation of CXCL10 in breast cancer 4T1 cells. Exp Mol Med. 2017 Feb. 17; 49(2):e295. doi: 10.1038/emm.2016.148.
13. Hayden M S, Ghosh S. NF-κB in immunobiology. Cell Res. 2011 February; 21(2):223-44. doi: 0.1038/cr.2011.13. Epub 2011 Jan. 18.
14. Wendel M, Galani I E, Suri-Payer E, Cerwenka A. Natural killer cell accumulation in tumors is dependent on IFN-gamma and CXCR3 ligands. Cancer Res. 2008 Oct. 15; 68(20):8437-45. doi: 10.1158/0008-5472.CAN-08-1440.
15. Giovanni Bernardini, Fabrizio Antonangeli, Valentina Bonanni, and Angela Santoni. Dysregulation of Chemokine/Chemokine Receptor Axes and NK Cell Tissue Localization during Diseases Front Immunol. 2016; 7: 402. Published online 2016 Oct. 6. doi: 10.3389/fimmu.206.00402.
16. Datta A, Kim H, McGee L, Johnson A E, Talwar S, et al., High-throughput screening identified selective inhibitors of exosome biogenesis and secretion: A drug repurposing strategy for advanced cancer. Sci Rep. 2018 May 25; 8(1):8161. doi: 10.1038/s41598-018-26411-7.
17. Z. Wang, J. C. Valera, X. Zhao, Q. Chen, J. Silvio Gutkind, mTOR co-targeting strategies for head and neck cancer therapy. Cancer Metastasis Rev 36, 491-502 (2017).
18. R. L. Ferris, Immunology and Immunotherapy of Head and Neck Cancer. J Clin Oncol 33, 3293-3304 (2015).
19. G. L. Beatty, W. L. Gladney, Immune escape mechanisms as a guide for cancer immunotherapy. Clin Cancer Res 21, 687-692 (2015).
20. Bae S, Brumbaugh J, Bonavida B., Exosomes derived from cancerous and non-cancerous cells regulate the anti-tumor response in the tumor microenvironment. Genes Cancer. 2018 March; 9(3-4):87-100. doi: 10.18632/genesandcancer.172.
21. Yang H., Sun L., Mao Y., The role of exosomes in tumor immunity. Ann Transl Med. 2018 December; 6(Suppl 2):S 16. doi: 10.21037/atm.2018.12.03.

1. A method of treating a tumor in a subject comprising administering to a subject having a tumor in need thereof an effective amount of a pharmaceutically acceptable composition comprising an agent that inhibits CHMP2A gene expression or function, or the endosomal sorting complex required for transport (ESCRT), in a cell of the tumor.

2. The method of claim 1, wherein the agent inhibits transcription or translation of CHMP2A gene.

3. The method of claim 2, wherein the agent is a shRNA that inhibits CHMP2A gene expression.

4. The method of claim 1, wherein the agent inhibits ESCRT.

5. The method of claim 4, wherein the agent is a farnesyltransferase inhibitor, including tipifarnib.

6. The method of claim 1, wherein the administration increases tumor sensitivity to natural killer cells or T cells.

7. The method of claim 6, wherein the tumor is a glioblastoma, meningioma or head and neck squamous cell carcinoma (HNSCC).

8. A method of treating a tumor in a subject comprising administering to a subject having a tumor in need thereof an effective amount of a pharmaceutically acceptable composition comprising an agent that inhibits gene expression or function of CHMP2A, PTPN9, ACER1, SNX7, MSR1, ANKRD46, IFT81, PLEKHF2, TRMT10A, or NARS in a cell of the tumor.

9. The method of claim 8, wherein the agent inhibits transcription or translation of CHMP2A gene.

10. The method of claim 9, wherein the agent is a shRNA that inhibits CHMP2A gene expression.

11. The method of claim 8, wherein the agent inhibits transcription or translation of PTPN9 gene.

12. The method of claim 8, wherein the agent inhibits ESCRT.

13. The method of claim 12, wherein the agent is a farnesyltransferase inhibitor, including tipifarnib.

14. The method of claim 8, wherein the administration increases tumor sensitivity to natural killer cells.

15. The method of claim 14, wherein the tumor is a glioblastoma, meningioma or head and neck squamous cell carcinoma (HNSCC).

16. A pharmaceutical composition comprising an agent that inhibits CHMP2A gene expression or function, or the endosomal sorting complex required for transport (ESCRT), in a cell of the tumor for use in treating the tumor in a patient in need thereof.

17. A pharmaceutical composition comprising an agent that inhibits gene expression or function of CHMP2A, PTPN9, ACER1, SNX7, MSR1, ANKRD46, IFT81, PLEKHF2, TRMT10A, and NARS, in a cell of a tumor for use in treating the tumor in a subject in need thereof.

18. The composition of claim 16, wherein the tumor cell is a glioblastoma, meningioma, or head and neck squamous cell carcinoma (HNSCC).

19. A method of increasing cell-mediated killing immune response capacity of natural killer (NK) cells in a subject in need thereof, comprising administering to the subject an effective amount of an agent that inhibits gene expression or function of CHMP2A, PTPN9, ACER1, SNX7, MSR1, ANKRD46, IFT81, PLEKHF2, TRMT10A, and NARS.

20. The composition of claim 19, wherein the subject has a tumor selected from a glioblastoma, meningioma, or head and neck squamous cell carcinoma (HNSCC).
